(19) United States
(12) Patent Application Publication
(10) Pub. No.: US 2023/0355474 A1
(43) Pub. Date: Nov. 9, 2023

(54) MEDICAL FLUID WARMING JACKET FOR AN IV FLUID BAG AND METHOD FOR WARMING AN IV FLUID BAG

(71) Applicants: John J. Sandoval, Waxhaw, NC (US); Benjamin Lorenzo Gatti, Indian Trail, NC (US); Raeshon McNeil, Charlotte, NC (US)

(72) Inventors: John J. Sandoval, Waxhaw, NC (US); Benjamin Lorenzo Gatti, Indian Trail, NC (US); Raeshon McNeil, Charlotte, NC (US)

(21) Appl. No.: 17/634,345

(22) PCT Filed: Sep. 10, 2021
(86) PCT No.: PCT/US21/49847

§ 371 Date: Feb. 10, 2022

Related U.S. Application Data
(60) Provisional application No. 63/076,581, filed on Sep. 10, 2020.

(51) Int. Cl. A61J 1/16; A61J 1/10; A61J 1/18

(52) U.S. Cl. CPC A61J 1/16; A61J 1/10; A61J 1/18; A61J 2200/42; A61J 2200/76; A61J 2200/72

(57) ABSTRACT

A medical fluid warming jacket for an IV fluid bag has a flexible jacket body including an integrally formed warming panel and window panel. The warming panel incorporates an electric heating element and at least one temperature sensor configured to measure a temperature of fluid contained within the IV bag. The window panel defines at least one see-through viewing area. An electronic control unit is operatively connected to the heating element and the temperature sensor, and includes a user interface and display. The electronic control unit takes input from the user interface to control the heating element and cooperates with the temperature sensor to output a measured temperature to the display.

### TECHNICAL FIELD AND BACKGROUND OF THE DISCLOSURE

The present disclosure relates broadly and generally to a medical fluid warming jacket for an intravenous (IV) fluid bag and method for warming an IVfluid bag.

There are many known benefits to maintaining normothermia to patients receiving intravenous fluids. Infusion of IV fluids at the right temperature partly influences clinical outcomes of critically ill patients. Because of this, there are several products that are designed to warm fluids prior to or during administration. The problem with warming incubators is that once the bag of fluid is removed and placed at room temperature, it immediately begins to cool and is no longer at ideal temperature. Furthermore, the problem with typical fluid warmers is that supplemental tubing must be connected and primed and then the fluid must be passed through a warming device. This process is cumbersome and the disposable tubing and heating device are expensive precluding its widespread use in the hospital and routine operating room setting.

### SUMMARY OF EXEMPLARY EMBODIMENTS

Various exemplary embodiments of the present disclosure are described below. Use of the term “exemplary” means illustrative or by way of example only, and any reference herein to “the invention” is not intended to restrict or limit the invention to exact features or steps of any one or more of the exemplary embodiments disclosed in the present specification. References to “exemplary embodiment,” “one embodiment,” “an embodiment,” “various embodiments,” and the like, may indicate that the embodiment(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment, although they may.

It is also noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

According to one exemplary embodiment, the present disclosure comprises a medical fluid warming jacket for an IV fluid bag. The fluid warming jacket has a flexible jacket body including an integrally formed warming panel and window panel. The warming panel incorporates an electric heating element and at least one temperature sensor configured to measure a temperature of fluid contained within the IV bag. The temperature sensor may measure fluid temperature inside the IV bag either indirectly using one or more contact sensors touching the jacket body, or directly using internal probe sensors or the like, or directly using no-contact (e.g, infrared) sensors. The window panel defines at least one see-through viewing area. An electronic control unit is operatively connected to the heating element and the temperature sensor, and includes a user interface and display (e.g., LCD screen). The electronic control unit takes input from the user interface to control the heating element and cooperates with the temperature sensor to output a measured temperature to the display.

According to another exemplary embodiment, the electronic control unit further comprises a microprocessor and random-access memory. In this embodiment, the exemplary fluid warming jacket may be programmable, and may further incorporate other electronics such as a transmitter, transceiver, modem (e.g., Bluetooth modem) or the like for wirelessly transmitting realtime information such as fluid temperature, bag volume, infusion rate and other data to remote devices (e.g., smartphone, desktop computer, cloud storage, and the like).

According to another exemplary embodiment, the electronic control unit comprises an electronic thermostat.

According to another exemplary embodiment, the temperature sensor comprises a plurality of spaced-apart contact sensors located at measurement points within the warming panel of the jacket body. The exemplary contact sensors use conduction to monitor changes in temperature of the IV fluid bag.

According to another exemplary embodiment, the temperature sensor comprises at least one no-contact sensor. In this embodiment, the no-contact sensor may use convection and radiation to monitor changes in temperature within the IV fluid bag. Whether contact or no-contact, the exemplary temperature sensor may be electro-mechanical, resistive or electronic.

According to another exemplary embodiment, the temperature sensor comprises an infrared thermometer.

According to another exemplary embodiment, the jacket body incorporates a fluid volume sensor.

According to another exemplary embodiment, the fluid volume sensor includes a plurality of spaced-apart capacitive sensors located at measurement points within the warming panel of the jacket body. Alternatively, weight measurements may be used to determine fluid volume within the IV bag.

According to another exemplary embodiment, the display indicates a volume of fluid inside the IV fluid bag.

According to another exemplary embodiment, the window panel is configured to wraparound the IV fluid bag and releasably attach to the warming panel.

According to another exemplary embodiment, the jacket body has flexible first and second wraparound fastener panels including complementary patches of releasable hook and loop fasteners. Alternatively, a single flexible fastener panel comprising one of hook or loop fasteners may releasably attach to a single complementary patch of fasteners locating on either the warming panel or window panel.

According to another exemplary embodiment, the jacket body is constructed of a thermal insulating material.

According to another exemplary embodiment, a rechargeable battery is operatively connected to the control unit for supplying electrical power to integrated components of the medical fluid warming jacket.

According to another exemplary embodiment, the display indicates a charge status of the battery.

According to another exemplary embodiment, an upper bag support is located proximate a top edge of the jacket body and is adapted for hanging a relatively large size (e.g., 1000 ml) collapsible fluid bag to be held inside the jacket body. The term “upper bag support” refers broadly herein to any structure, such as a J-hook, S-hook, V-hook or the like, capable of supporting the weight of the IV bag inside the flexible body of the fluid warming jacket. The exemplary support may comprise a length of material (e.g., metal or plastic) having a curved or indented portion, such that it can be used to grab onto, connect, or otherwise attach itself onto another object.

According to another exemplary embodiment, a lower bag support is located a spaced distance below the upper bag support and is adapted for hanging a relatively small size (e.g., 500 ml) collapsible fluid bag to be held inside the jacket body. Like the term “upper bag support”, the “lower bag support” refers broadly herein to any structure, such as a J-hook, S-hook, V-hook or the like, capable of supporting the weight of the smaller IV bag inside the flexible body of the fluid warming jacket. The exemplary support may comprise a length of material (e.g., metal or plastic) having a curved or indented portion, such that it can be used to grab onto, connect, or otherwise attach itself onto another object.

According to another exemplary embodiment, the lower bag support is configured to reside substantially flat in a stowed condition. In exemplary embodiments, the bag support may comprise a swivel or pivoted hook.

According to another exemplary embodiment, the warming panel is divided into multiple independently controllable warming zones.

According to another exemplary embodiment, the warming zones comprise an upper zone occupying substantially an upper half of the warming panel and a lower zone occupying substantially a lower half of the warming panel.

According to another exemplary embodiment, a hanger is located at a top of the jacket body to vertically hang the warming jacket from an IV bag pole. The term “hanger” is broadly defined herein to mean any structure or the absence of structure (e.g., hole or other opening) which can be used to suspend the IV bag vertically from the bag pole.

### BRIEF DESCRIPTION OF THE DRAWINGS

Exemplary embodiments of the present disclosure will hereinafter be described in conjunction with the following drawing figures, wherein like numerals denote like elements, and wherein:

FIG. 1 is an environmental view of the exemplary fluid warming jacket applied to a collapsible IV fluid bag and hung from a standard vertical IV pole;

FIG. 2 is a further perspective view of the exemplary fluid warming jacket applied to a standard small size (500 ml) IV fluid bag, and separately illustrating both the large size (1000 ml) fluid bag and the small size fluid bag;

FIGS. 3 and 4 are perspective front and back views of the exemplary fluid warming jacket in a partially open condition;

FIGS. 5, 6, and 7 are views of the exemplary fluid warming jacket in a fully open (substantially flat) condition;

FIG. 8 is a diagram illustrating exemplary overlying components of the composite warming panel of the present fluid warming jacket;

FIG. 9 is a further diagram illustrating heating elements and temperature and capacitive sensors of the exemplary warming panel;

FIGS. 10 and 11 are views of the exemplary fluid warming jacket in an open condition, and illustrating selective activation of the upper and lower warming zones;

FIG. 12 is a diagrammatic view of the electronic control unit incorporated in the exemplary fluid warming jacket;

FIGS. 13 and 14 are views of an exemplary wall-mounted charging station used to recharge an integrated battery of the present fluid warming jacket; and

FIG. 15 is a further view showing an alternative means for recharging the battery incorporated in the present fluid warming jacket.

### DESCRIPTION OF EXEMPLARY EMBODIMENTS AND BEST MODE

The present invention is described more fully hereinafter with reference to the accompanying drawings, in which one or more exemplary embodiments of the invention are shown. Like numbers used herein refer to like elements throughout. This invention may, however, be embodied in many different forms and should not be construed as limited to the embodiments set forth herein; rather, these embodiments are provided so that this disclosure will be operative, enabling, and complete. Accordingly, the particular arrangements disclosed are meant to be illustrative only and not limiting as to the scope of the invention, which is to be given the full breadth of the appended claims and any and all equivalents thereof. Moreover, many embodiments, such as adaptations, variations, modifications, and equivalent arrangements, will be implicitly disclosed by the embodiments described herein and fall within the scope of the present invention.

Although specific terms are employed herein, they are used in a generic and descriptive sense only and not for purposes of limitation. Unless otherwise expressly defined herein, such terms are intended to be given their broad ordinary and customary meaning not inconsistent with that applicable in the relevant industry and without restriction to any specific embodiment hereinafter described. As used herein, the article “a” is intended to include one or more items. Where only one item is intended, the term “one”, “single”, or similar language is used. When used herein to join a list of items, the term “or” denotes at least one of the items, but does not exclude a plurality of items of the list.

For exemplary methods or processes of the invention, the sequence and/or arrangement of steps described herein are illustrative and not restrictive. Accordingly, it should be understood that, although steps of various processes or methods may be shown and described as being in a sequence or temporal arrangement, the steps of any such processes or methods are not limited to being carried out in any particular sequence or arrangement, absent an indication otherwise. Indeed, the steps in such processes or methods generally may be carried out in various different sequences and arrangements while still falling within the scope of the present invention.

Additionally, any references to advantages, benefits, unexpected results, or operability of the present invention are not intended as an affirmation that the invention has been previously reduced to practice or that any testing has been performed. Likewise, unless stated otherwise, use of verbs in the past tense (present perfect or preterit) is not intended to indicate or imply that the invention has been previously reduced to practice or that any testing has been performed.

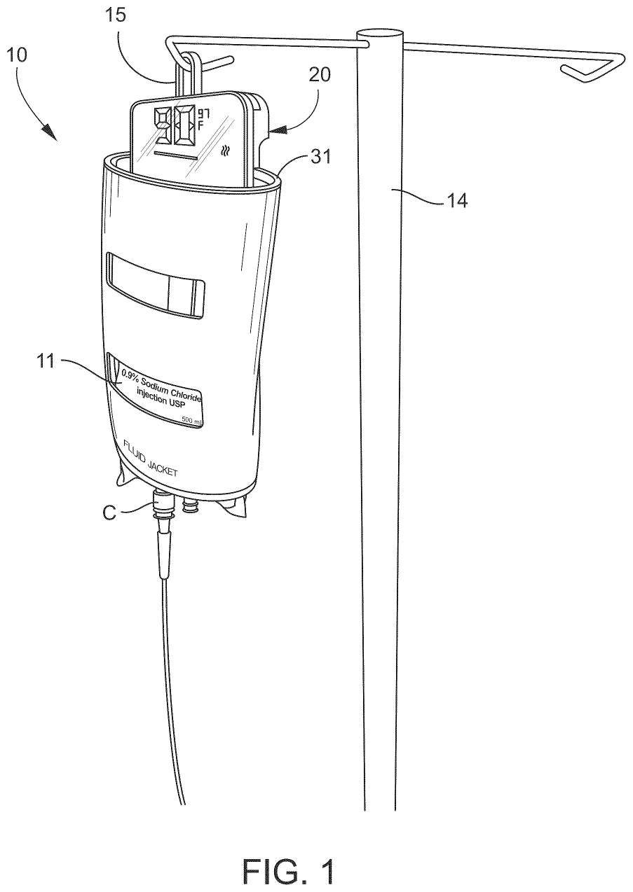

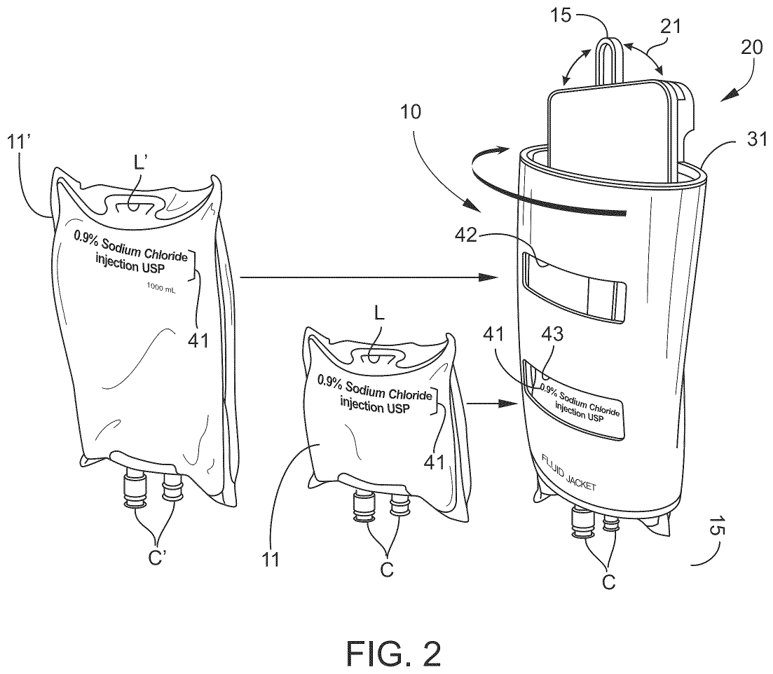

Referring now specifically to the drawings, a medical fluid warming jacket according to one exemplary embodiment of the present disclosure is illustrated in FIGS. 1 and 2, and show generally at broad reference numeral 10. In exemplary embodiments, the fluid warming jacket 10 is configured to receive and hold a standard collapsible intravenous (IV) fluid bag 11, 11′, including both a standard 500 milliliter (ml) bag and a standard 1000 milliliter (ml) bag, respectively. IV bags typically contain a medical fluid, such as sodium chloride, and are conventionally manufactured of a medical grade plastic—most often made from polyvinyl chloride (PVC). The IV bag 11, 11′ is held by the warming jacket 10, as discussed further below, and is suspended from a standard IV pole 14 by a pivoted looped hanger 15 to gravity feed fluid from the bag 11, 11′ to a patient. The looped hanger 15 is attached to an electronic control unit 20 of the warming jacket 10, and pivots as indicated by arrows 21 between a stowed (hidden) position and an extended in-use position.

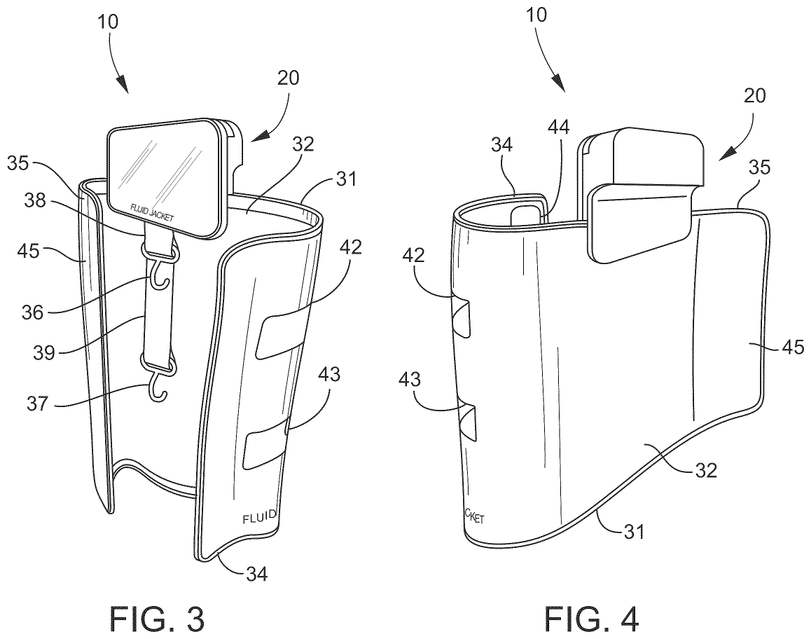

As best shown in FIGS. 2-7, the exemplary warming jacket 10 includes a flexible jacket body 31 with an integrally formed warming panel 32, window panel 33, and wraparound fastener panels 34, 35. The control unit 20, discussed further below, is affixed to a top edge of the jacket body 31 and houses electronic components of the warming jacket 10. Upper and lower tethered bag hooks 36, 37 are located at respective distal ends of nylon straps 38, 39 attached to the jacket body 31 and residing at predetermined suspension points adjacent an inside surface of the warming panel 32. The upper bag hook 36 inserts through an integrated loop L′ formed with a top edge of the large size (e.g., 1000 ml) collapsible fluid bag 11′ and locates the large fluid bag 11′ such that its entire length extends substantially from a top of the warming panel 32 to a bottom of the warming panel 32. The lower bag hook 37 likewise inserts through an integrated loop L formed with a top edge of the collapsible small size (e.g., 500 ml) fluid bag 11 and locates the small fluid bag 11 such that its entire length extends substantially from a midpoint of the warming panel 32 to the bottom of the warming panel 32. When hanging the large size fluid bag 11′, the lower hook 37 swivels to a stowed position substantially flat against the inside of warming panel 32 to avoid interfering with direct surface-to-surface contact between the fluid bag 11′ and warming panel 32.

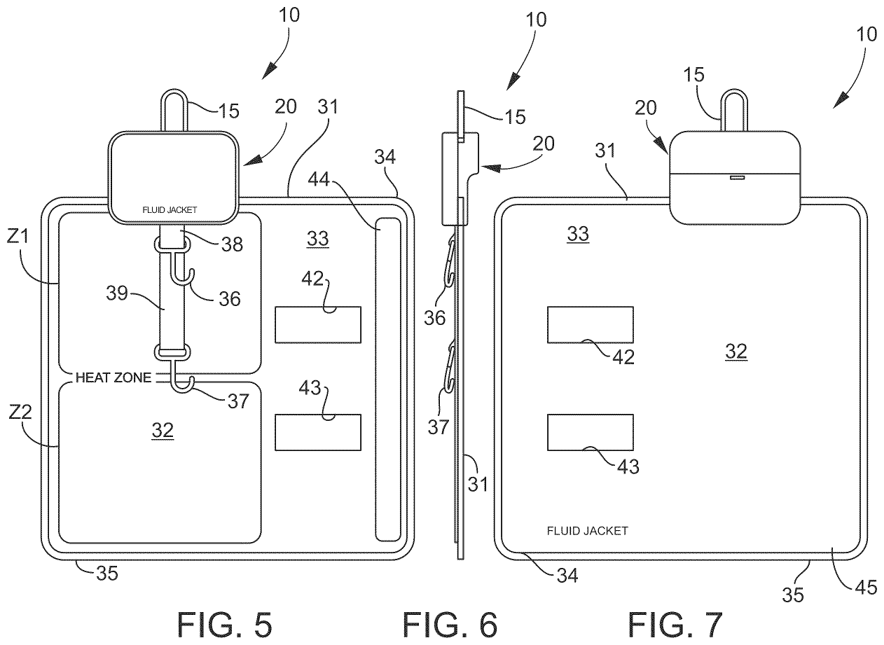

Once one of the small size or large size fluid bags 11, 11′ is placed on bag hook 36, 37 of the open warming jacket 10, the integrally-formed window panel 33 of jacket body 31 is folded over the fluid bag 11, 11′ such that certain bag indicia 41, including fluid type and concentration, located on labeled bag wall properly aligns with one of two see-through viewing areas 42, 43 formed with the window panel 33. Other viewing areas (not shown) may be formed with the window panel 33 for readily viewing and monitoring the volume of fluid inside the IV bag 11, 11′. The wraparound fastener panels 34, 35 releasably secure the warming jacket 10 around the fluid bag 11, 11′ (See FIG. 2) using complementary hook and loop fasteners, such as VELCRO®. In an exemplary embodiment, a vertical strip 44 of hook fasteners is located along an inside margin of the first fastener panel 34, while a complementary vertical strip 45 of look fasteners is located along an outside margin of the second fastener panel 35. The exemplary hook and look fasteners cooperate to releasably wrap the warming jacket 10 around the fluid bag 11, 11′ such that the non-labeled bag wall closely engages the inside surface of warming panel 32. The bottom of the wrapped warming jacket 10 remains open for ready access to bag tube connectors C. Exemplary dimensions of the present warming jacket 10 in an open condition are indicated in FIGS. 5, 6, and 7.

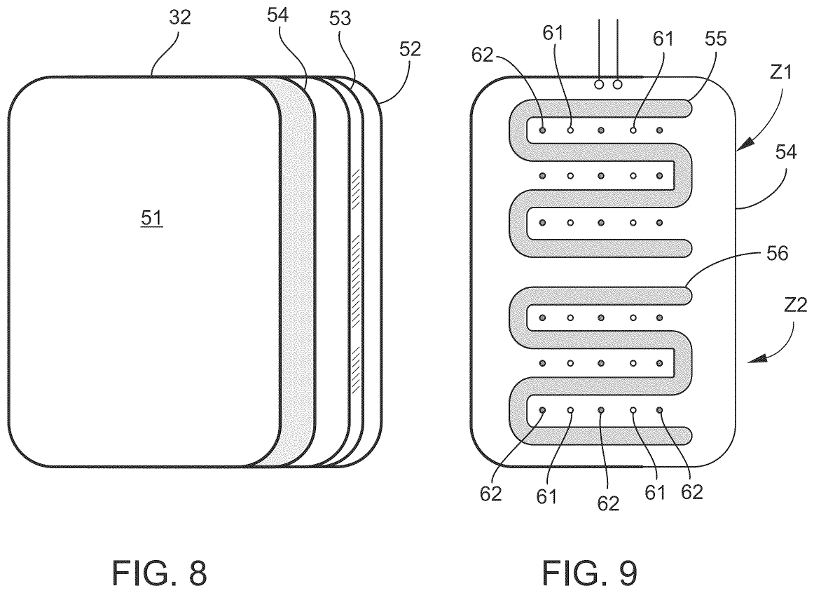

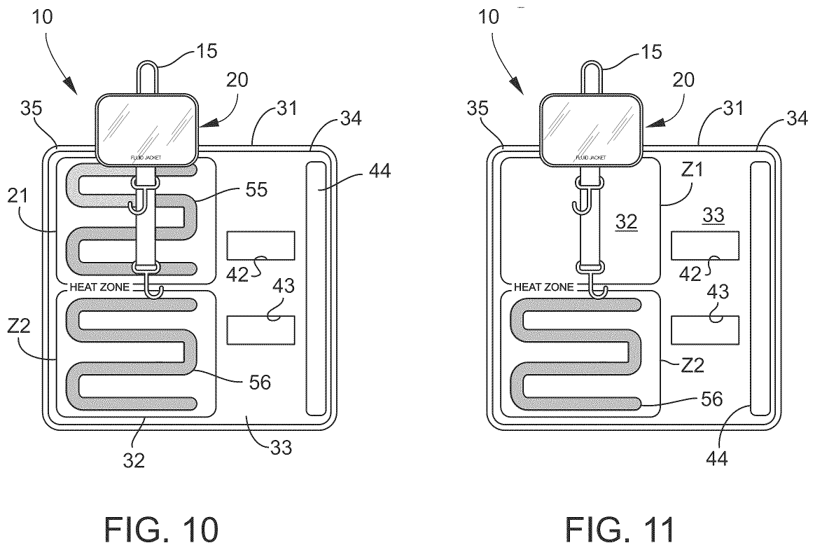

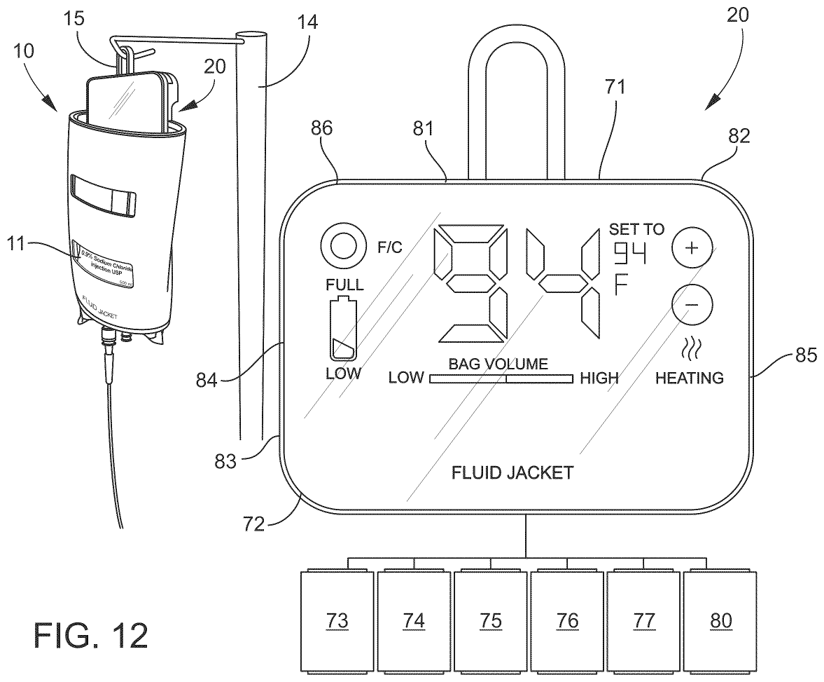

Referring to FIGS. 8-11, the warming panel 32 of the exemplary jacket 10 comprises a multi-layer composite including inside and outside fabric (e.g., nylon) layers 51, 52, an insulation layer 53, and a dual-sensing flexible printed heat circuit (PCB) 54. The exemplary heat circuit 54 comprises integrated heating elements 55, 56 dividing the warming panel 32 of jacket body 31 into upper and lower independently controllable warming zones Z1 and Z2. The upper warming zone Z1 occupies substantially an entire upper half of the warming panel 32 and the lower warming zone Z2 occupies substantially an entire lower half of the warming panel 32. Heating elements 55, 56 in both the upper and lower zones Z1, Z2 may be selectively activated, as represented in FIG. 10, when warming fluid contained in the large size IV fluid bag 11′, while the upper zone Z1 may be deactivated and only the lower zone Z2 activated, as represented in FIG. 11, when warming fluid contained in the small size IV fluid bag 11. Additionally, when warming a large size fluid bag 11′, as fluid volume drops below a predetermined threshold, the upper Z1 may automatically deactivate while the lower zone Z2 remains active, continuing to warm fluid in the lower portion of the bag. The exemplary heating elements 55, 56 may comprise resin-impregnated thermal coils. Other devices for electrically warming flexible panels exist in the prior art. One such device is disclosed in a published Korean Patent KR100249352B1. The complete disclosure of this prior patent is incorporated herein by reference.

In an exemplary embodiment, the present heat circuit 54 incorporates a number of temperature sensors 61 and fluid volume (e.g., capacitive) sensors 62 spaced apart and strategically located at predetermined measurement points within the upper and lower warming zones Z1, Z2 of warming panel 32. The temperature sensors 61 function to measure temperature of fluid contained within the IV bag 11, 11′, and the delivery temperature of the warmed fluid transferred from the bag 11, 11′ to the patient. The exemplary temperature sensors 61 may comprise contact sensors which directly (or indirectly through fabric layer) touch the IV bag 11, 11′, and which use conduction to monitor changes in temperature of the medical fluid. Alternatively, or in addition, the warming jacket 10 may be configured to utilize one or more temperature probe sensors to operatively “contact” the medical fluid, or one or more no-contact (e.g, infrared) sensors or thermometer. Exemplary no-contact sensors may use convection and radiation to monitor changes in temperature within the fluid bag 11, 11′. Whether contact or no-contact, the exemplary temperature sensors may be electro-mechanical, resistive or electronic.

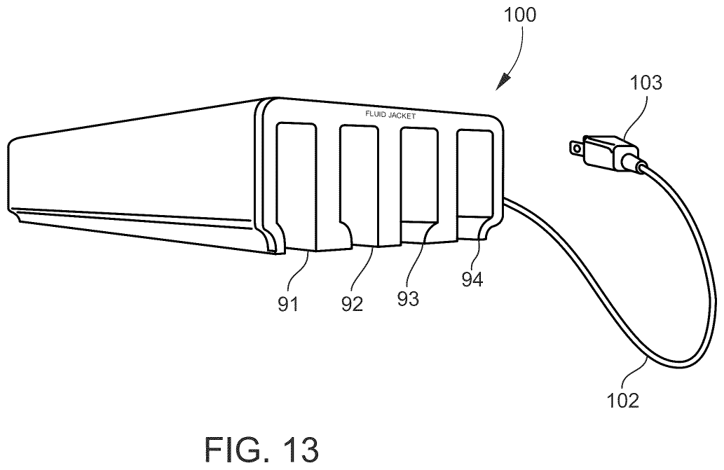

Referring to FIG. 12, the exemplary control unit 20 comprises a microcontroller operatively connected to (and communicated with) all integrated components of the warming jacket 10, including the printed dual-sensing heating circuit 54, the temperature sensors 61, and capacitive sensors 62 discussed above. In exemplary embodiments, control unit 20 comprises a touchscreen interface 71 and output display 72 (e.g., LCD screen), a microprocessor 73, RAM 74, data storage 75, transceiver 76, modem 77, and rechargeable battery 80. The rechargeable battery 80 is operatively connected to the control unit 20 and functions to supply electrical power to integrated components of the warming jacket 10. The battery 80 may comprise any suitable rechargeable battery known and used in the industry. One exemplary battery may comprise a rechargeable lithium ion battery.

The electronic control unit 20 takes input from the touchscreen interface 71 (i.e., a set-to temperature entered by the operator) to control the heating circuit 54, and communicates with the temperature sensors and capacitive sensors to output fluid data to the LCD display 72. The exemplary control unit 20 may be programmable, and may be configured to wirelessly transmit realtime information such as fluid temperature, bag volume, infusion rate and other data to remote devices (e.g., smartphone, desktop computer, cloud storage, and the like). The exemplary LCD display 72 may include a current measured fluid temperature 81, a set-to fluid temperature 82, a fluid volume indicator 83, a charge status of the battery 84, and a status of the heating circuit 85. The control unit 20 may be powered-on by push button 86. Once powered-on, a second push of power button 86 may convert the temperature display from Fahrenheit Celsius. Buttons 88 and 89 may be used to adjust the set-to temperature of the control unit 20. In the event fluid temperature exceeds the set-to temperature entered by the user, or in the event a prescribed maximum fluid temperature is reached, the control device 20 may emit an audio and/or visual alert. In alternative embodiments, the control unit 20 may comprise an electronic thermostat which communicates with temperature sensors to read and adjust the temperature of the warming panel.

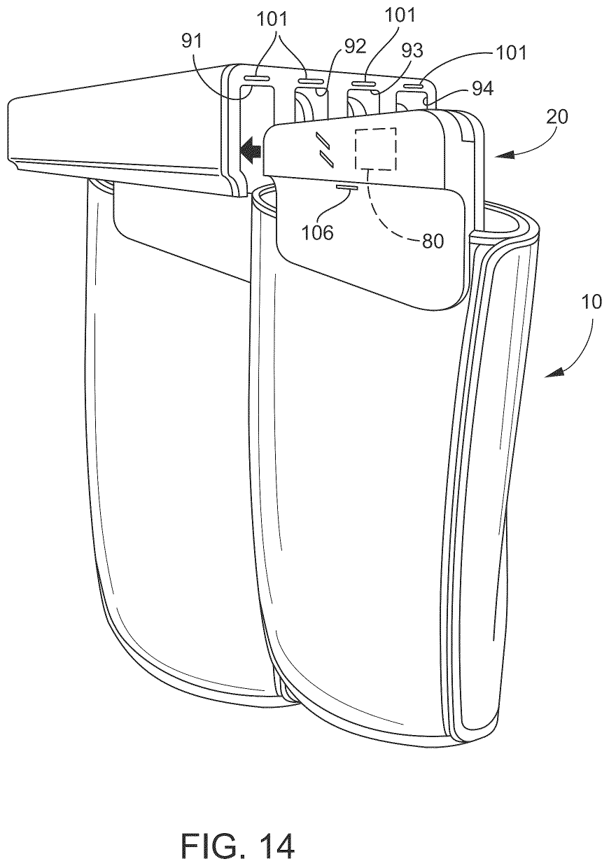

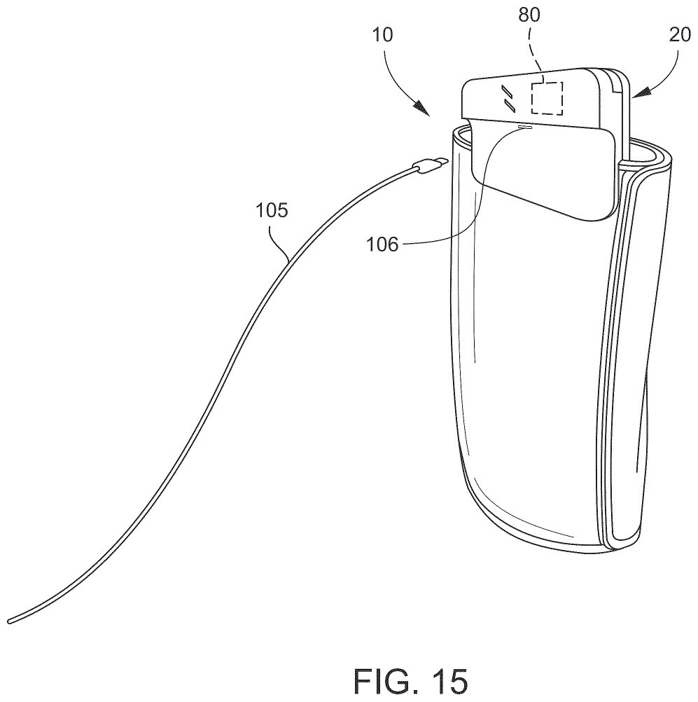

Referring to FIGS. 13, 14, and 15, in one embodiment the exemplary control unit 20 of warming jacket 10 is configured to slide into a selected one of a plurality of inductive charging channels 91, 92, 93, 94 of a wall-mounted charging station 100. The charging channels 91-94 use electromagnetic induction to transfer electricity to the integrated battery 80 of the control unit 20. Once the battery 80 is fully recharged, an indicator light 101 illuminates on the charging station 100 proximate the particular channel 91-94. For example, a green indicator light 101 may indicate a fully charged battery, a yellow indicator light 101 a partially charged battery, and a red indicator light 101 an uncharged battery. The charging station 100 may used to simultaneous charge multiple warming jackets 10 and may be powered using an operatively attached power cord 102 with plug 103 and a standard AC wall outlet. Alternatively, the battery 80 of warming jacket 10 may be charged using a standard plug-in power cord 105 and charging port 106 shown in FIG. 15.

Although not shown, the exemplary warming jacket 10 may incorporate, or may be used in combination with, an inflatable bladder applicable for applying controlled pressure to the IV fluid bag in order to speed the transfer of warmed fluid to the patient. In this embodiment, the warming jacket or bladder may incorporate a pressure monitor to ensure that the pressure exerted upon the fluid bag is not excessive and that the fluid administration rate is not within a dangerous range.

For the purposes of describing and defining the present invention it is noted that the use of relative terms, such as “substantially”, “generally”, “approximately”, and the like, are utilized herein to represent an inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

Exemplary embodiments of the present invention are described above. No element, act, or instruction used in this description should be construed as important, necessary, critical, or essential to the invention unless explicitly described as such. Although only a few of the exemplary embodiments have been described in detail herein, those skilled in the art will readily appreciate that many modifications are possible in these exemplary embodiments without materially departing from the novel teachings and advantages of this invention. Accordingly, all such modifications are intended to be included within the scope of this invention as defined in the appended claims.

In the claims, any means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. Unless the exact language “means for” (performing a particular function or step) is recited in the claims, a construction under 35 U.S.C. § 112(f) [or 6th paragraph/pre-AIA] is not intended. Additionally, it is not intended that the scope of patent protection afforded the present invention be defined by reading into any claim a limitation found herein that does not explicitly appear in the claim itself.

What is claimed:

1. A medical fluid warming jacket for an IV fluid bag, comprising:
a flexible jacket body comprising an integrally formed warming panel and window panel, said warming panel incorporating an electric heating element and at least one temperature sensor configured to measure a temperature of fluid contained within the IV bag, and wherein said window panel defines at least one see-through viewing area;
an electronic control unit operatively connected to said heating element and said temperature sensor, and comprising a user interface and display; and
whereby said electronic control unit takes input from said user interface to control said heating element and cooperates with said temperature sensor to output a measured temperature to said display.

2. The medical fluid warming jacket according to claim 1, wherein said electronic control unit further comprises a microprocessor and random-access memory.

3. The medical fluid warming jacket according to claim 1, wherein said electronic control unit comprises an electronic thermostat.

4. The medical fluid warming jacket according to claim 1, wherein said temperature sensor comprises a plurality of spaced-apart contact sensors located at measurement points within said warming panel of said jacket body.

5. The medical fluid warming jacket according to claim 1, wherein said temperature sensor comprises at least one no-contact sensor.

6. The medical fluid warming jacket according to claim 1, wherein said temperature sensor comprises an infrared thermometer.

7. The medical fluid warming jacket according to claim 1, and comprising a fluid volume sensor adapted for measuring a volume of fluid contained within the IV bag.

8. The medical fluid warming jacket according to claim 7, wherein said fluid volume sensor comprises a plurality of spaced-apart capacitive sensors located at measurement points within said warming panel of said jacket body.

9. The medical fluid warming jacket according to claim 1, wherein said display indicates a volume of fluid inside the IV fluid bag.

10. The medical fluid warming jacket according to claim 1, wherein said window panel is configured to fold over the IV fluid bag such that indicia applied to the IV fluid bag is visible through said see-through viewing area.

11. The medical fluid warming jacket according to claim 1, wherein said jacket body has first and second fastener panels comprising complementary patches of releasable hook and loop fasteners.

12. The medical fluid warming jacket according to claim 1, wherein said jacket body is constructed of a thermal insulating material.

13. The medical fluid warming jacket according to claim 1, and comprising a rechargeable battery for supplying electrical power to integrated components of said medical fluid warming jacket.

14. The medical fluid warming jacket according to claim 13, wherein said display indicates a charge status of said battery.

15. The medical fluid warming jacket according to claim 1, and comprising an upper bag support located proximate a top edge of said jacket body and adapted for hanging a relatively large size fluid bag to be held inside said jacket body.

16. The medical fluid warming jacket according to claim 15, and comprising a lower bag support located a spaced distance below said upper bag support and adapted for hanging a relatively small size fluid bag to be held inside said jacket body.

17. The medical fluid warming jacket according to claim 16, wherein said lower bag support is configured to reside substantially flat in a stowed condition.

18. The medical fluid warming jacket according to claim 1, wherein said warming panel is divided into multiple independently controllable warming zones.

19. The medical fluid warming jacket according to claim 18, wherein said warming zones comprise an upper zone occupying substantially an upper half of said warming panel and a lower zone occupying substantially a lower half of said warming panel.

20. The medical fluid warming jacket according to claim 1, and comprising a hanger located at a top of said jacket body to vertically hang said warming jacket from an IV bag pole.
